(19) United States
(12) Patent Application Publication
(10) Pub. No.: US 2026/0083816 A1
(43) Pub. Date: Mar. 26, 2026

(54) METHODS OF TREATING CANCER BY TARGETING DRIVERS

(71) Applicant: DELTA NEXT-GENE, LLC, Santa Monica, CA (US)

(72) Inventors: Erlinda M. Gordon, Santa Monica, CA (US); Fredrick L. Hall, Santa Monica, CA (US)

(21) Appl. No.: 18/923,976

(22) Filed: Oct. 23, 2024

Related U.S. Application Data
(63) Continuation of application No. PCT/US2023/019741, filed on Apr. 25, 2023.
(60) Provisional application No. 63/334,347, filed on Apr. 25, 2022.

(51) Int. Cl. A61K 38/16; A61K 31/4196; A61K 39/00; A61K 39/395; A61K 48/00; A61P 35/00

(52) U.S. Cl. CPC A61K 38/16; A61K 31/4196; A61K 39/39558; A61K 48/0058; A61P 35/00; A61K 2039/505

(57) ABSTRACT

The present disclosure provides methods of using combination therapies to treat cancer. These methods are based on inhibiting cyclin proteins along with proteins along the Cyclin Axis. Thus, a method of the disclosure may generally be practiced by administering to an individual having cancer a cytocidal inhibitor of cyclin, along with a therapeutic agent that targets other molecules involved in pathways that promote cyclin activity. These therapeutic agents may target biomarkers on the surface of a cancer cell and that promote cyclin activity as well as intracellular proteins involved in pathways that affect, or are affected by, cyclin activity.

### CROSS-REFERENCE TO RELATED APPLICATIONS

This application is a continuation of International Patent Application No. PCT/US2023/019741, filed Apr. 25, 2023, and titled “METHODS OF TREATING CANCER BY TARGETING DRIVERS,” which in turn claims priority to U.S. Provisional Patent Application No. 63/334,347 , filed Apr. 25, 2022, and titled “METHODS OF TREATING CANCER BY TARGETING DRIVERS,” the contents of each of which are incorporated herein in their entirety.

### BACKGROUND

Cancer is a major public health and economic issue and the burden of this disease is expected to grow. 18 million cases of cancer were diagnosed in 2018, and this number is expected to reach 29 million by 2040, due to aging and growth of the population. Moreover, an estimated 43.8 million people were living with cancer in 2018, having been diagnosed in the previous five years.

Early treatments for cancer involved the use of chemicals (chemotherapy) that non-specifically killed rapidly dividing cells. While such treatments would kill cancer cells, they also caused discomforting short-term side effects, such as nausea and hair loss, and serious long-term side effects such as heart damage, nerve damage and fertility problems. Moreover, cancer cells often became resistant to chemotherapeutic agents.

More recent cancer therapies are based on exploiting more specific biological differences between cancer cells and non-cancer cells (aka, normal cells). Many of these therapies are based on the differential, or exclusive, expression of proteins in cancer cells but not in normal cells. For example, many cancer cells express proteins on their surfaces that are either not expressed or expressed to a lesser degree on the surface of normal cells. These surface proteins may also be used to identify cancer cells, and thus may be referred toa s biomarkers. In addition to aberrant protein expression, cancer cells express mutant proteins, many of which are involved in cell cycle control. Mutations in these proteins leads to loss of cell cycle control, resulting in unrestrained cell growth, which is a hallmark of cancer.

Breast cancer is the most common cancer diagnosis in women. Triple positive breast cancer is the second most common subtype of invasive breast cancer (IBC) with an age-adjusted rate of 13.4 new cases per 100,000 women, based on 2014-2018 cases. While established differences in basic biology, clinical performance, and treatment response between HER2+/HR+ and HER2+/HR− breast cancers are well-known, these two IBC categories are often treated essentially the same (Dieci 2020). This indistinguishable clinical management comes from the scarcity of clinical trials specifically designed for HER2+/HR+ or HER2 +/HR− patients. In light of current literature, these intrinsic differences between these breast cancer subtypes can no longer be ignored and call for clinical studies testing specific treatments for HER2+/HR+ or HER2+/HR− breast cancer patients. Moreover, there is increasing debate about the use of toxic chemotherapy as standard of care therapy for early-stage triple receptor positive IBC in post-menopausal women. (Johnston 2018) In the era of precision medicine, targeting oncogenic drivers along the executive signaling pathways of cell transformation and proliferation would be ideal, if not crucial, to improve treatment outcome parameters at a time when molecular profiling of tumors and detection of circulating tumor DNA have become increasing available in the clinic [Al Shihabi 2018, Hall 2021, Gordon 2021].

A family of proteins known to play a key role in control of the cell cycle is are the cyclins, which include, at least, cyclinG1 and cyclinD1.

Moreover, many proteins that have been discovered to be mutated in cancer cells have been found to either be affected by cyclinG1 or cyclinD1, or to affect the expression and/or activity of the cyclins, thereby forming a Cyclin Axes of influence. These axes represent promising targets for new anti-cancer therapies.

Strategically, targeting key oncogenic drivers along a Cyclin Axis, as well as targeting the tumor microenvironment (TNE), are concepts that have been formally validated in clinical trial, and long term survivors of chemo-resistant patients treated with a specific Cyclin G1 inhibitor have been reported. [Kim 2017 Al-Shihabi 2018, Liu 2021]. Specifically, DeltaRex-G—the first and, so far, only tumor-targeted gene expression vector of its kind—exhibits both precision tumor-targeting properties and selective cytocidal/anti-tumor properties, displaying a remarkable “single-agent” anti-cancer efficacy. (1) Structurally, the DeltaRex-G gene delivery vector displays a Signature (SIG)-binding peptide on its enveloped surface by design for targeting anaplastic collagenous Signature proteins abnormally exposed within the TME during cancer invasion/metastasis; (2) Genetically, the therapeutic payload encodes a dominant-negative Cyclin G1 inhibitor protein which blocks Cyclin G1 cell competence and survival function—thereby selectively eradicating a broad spectrum of tumors, including tumor-forming cells involved in microscopic and occult disease: cancer stem cells, tumor-associated fibroblasts (TAFs), and associated proliferative neovasculature [Hall 2021, Gordon 2021]. When injected intravenously, the DeltaRex-G nanoparticles seek out and accumulate in the TME where SIG proteins are abnormally exposed in the vicinity of proliferative cancer cells and tumor-associated cells, hence augmenting effective drug concentration [Hall 2000, 2010; Gordon 2000, 2001, 2010]. FIG. 1 is a graphic illustration of the DeltaRex-G gene vector. DeltaRex-G has been described in detail in US20190382459, which is hereby incorporated by reference in its entirety.

To date, DeltaRex-G has induced long term survival (>12 years) in chemotherapy-resistant patients presenting with advanced or metastatic pancreatic cancer, malignant peripheral nerve sheath tumor, osteosarcoma, breast cancer and B-cell lymphoma, as demonstrated in formal Phase 1 and Phase 2 US-based trials and in Philippine clinical trials [Liu 2021]). Based on demonstrations of unique safety and efficacy in these hard-to-treat Stage 4 cancers, DeltaRex-G has gained USFDA approval for the Blessed Protocol: Expanded access for DeltaRex-G for advanced pancreatic cancer and sarcoma for an intermediate size population (NCT04091295) [Blessed Protocol, 2020].

Moreover, results from a Phase 1/2 clinical trial using DeltaRex-G for chemotherapy resistant Stage 4 breast cancer were presented at the American Society of Gene and Cell Therapy Annual Meetings [Bruckner et al., 2019]. In this study, DeltaRex-G induced 17.6% response rate by PET/Choi criteria, 76% tumor control rate and 83% one-year survival rate, with no serious adverse events in patients receiving the highest dose level of DeltaRex-G. These data suggest that (1) DeltaRex-G is uniquely safe and exhibits antitumor activity. However, there still remains a population for which the results achieved using DeltaRex-G alone was not satisfactory. Combining DeltaRex-G with other targeted cancer therapies, particularly those along a Cyclin Axis, should provide improve treatment results. The present disclosure provides such treatments.

### DESCRIPTION OF THE DISCLOSURE

The present disclosure provides methods of using combination therapies to treat cancer. These methods are based on inhibiting cyclin proteins along with proteins along the Cyclin Axis. Thus, a method of the disclosure may generally be practiced by administering to an individual having cancer a cytocidal inhibitor of cyclin, along with a therapeutic agent that targets other molecules involved in pathways that promote cyclin activity. These therapeutic agents may target biomarkers on the surface of a cancer cell and that promote cyclin activity as well as intracellular proteins involved in pathways that affect, or are affected by, cyclin activity.

Before the present disclosure is further described, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. For example, a compound refers to one or more compound molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Publications discussed herein are referenced for their content. The publication dates may need to be independently confirmed. All publications mentioned herein are incorporated herein by reference to disclose and describe the methods and/or materials in connection with which the publications are cited.

### BRIEF DESCRIPTION OF THE DRAWINGS

FIG. 1 is a graphic illustration of the DeltaRex-G gene vector.

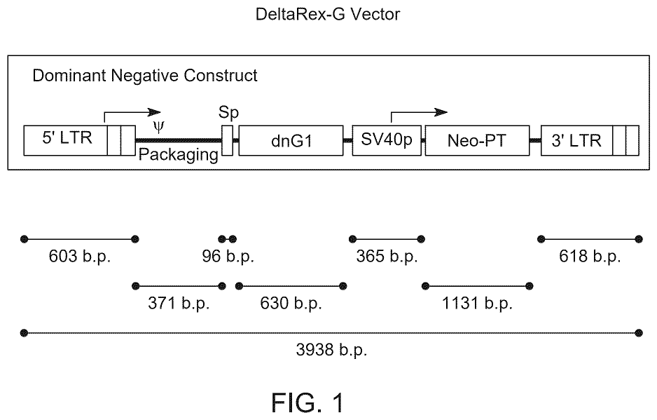

FIG. 2 is a graphic illustration of six proximal oncogenic mechanisms activating CCNG1.

FIG. 3 is a graphic illustration of CCNG1 pathways.

### DETAILED DESCRIPTION OF THE INVENTION

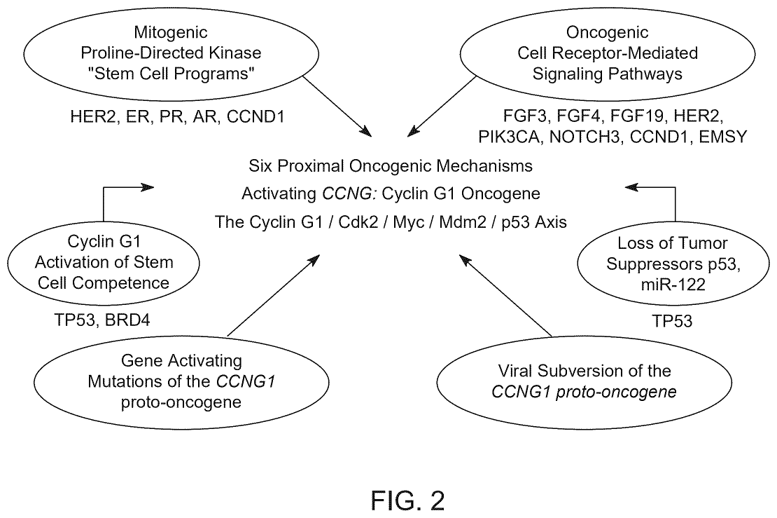

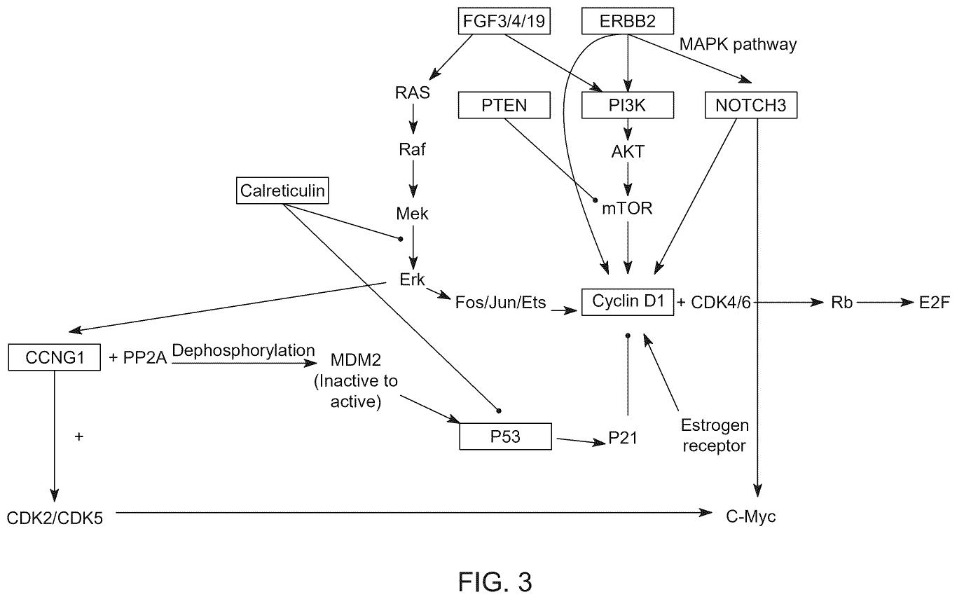

One aspect of the disclosure is a method of treating a cancer in an individual, comprising administering to the individual a first therapeutic agent comprising a tumor-targeted vector encoding a cytocidal inhibitor of cyclin 1, and a second therapeutic agent that acts on at least one molecular target in a biochemical pathway that increase cyclin protein activity, wherein the second therapeutic agent inhibits or reduces one or more activities or products of the biochemical pathway. In one aspect, cells of the cancer may express at least one biomarker that is part of the biochemical pathway that promotes cyclin protein activity. The at least one biomarker, may be expressed on the surface of a cancer cell. In one aspect, the molecular target of the second therapeutic agent may be the at least one biomarker. In some aspects, the second therapeutic agent may bind to the at least one biomarker, thereby affecting its activity and causing inhibition or reduction of one or more activities, or products, of the biochemical pathway. The biochemical pathway may comprise an oncogenic receptor-mediated signaling pathway, a tumor suppressor pathway, or a stem cell renewal or differentiation pathway (see FIGS. 2 and 3).

In some aspects, the at least one biomarker may be selected from the group consisting of estrogen receptor (ER), progesterone receptor (PR) human epidermal growth factor 2 (HER2), androgen receptor (AR), fibroblast growth factor 13 (FGF13), FGF14, FGF19, phosphatidylinositol 3-kinase catalytic subunit alpha (PIK3CA), NOTCH3, EMSY, tumor protein 53 (TP52), and bromodomain-containing protein 4 (BRD4). In some aspects, the second therapeutic agent acts on a biomarker selected from the group consisting of ER, PR HER2, AR, FGF13, FGF14, FGF19, PIK3CA, NOTCH3, EMSY, TP52, and BRD4.

Without being bound by theory, the following description of biomarkers having use in methods of the disclosure are provided so that the reader may gain understanding into possible biochemical pathways of the disclosure. HER2/neu stimulates the Ras-Raf-Mapk and PI3K pathways of intracellular signal transduction (FIG. 2). Erk activation by the Ras-Raf pathway leads to activation of the CCND1 promoter by Fos/Jun/Ets transcription factors. CCND1 makes a complex with Cdk4/6 to phosphorylate Rb and release E2F proteins that regulate G1-S transition. Activation of the PI3K-Akt pathway results in enhanced anti-apoptotic action through inhibition of the pro-apoptosis proteins (e.g., Bad, GSK3 and the transcription factor FKHR-L1. Further, activation of the JAK-STAT pathway by HER2 leads to cellular proliferation. A major mitogenic player acting downstream of HER2 is CCND1, promoting cell cycle progression. CCND1 also interacts with the ERα to promote its transcriptional activity in Cdk-independent fashion. [Taneja 2010].

FGF3/4/19 binds to FGFR2b or FGFR2c, respectively, with HSPG as a cofactor and induces the formation of ternary FGFs-FGFR2-HS complexes, which activates the FGFR2 intracellular tyrosine kinase domain by phosphorylation of specific tyrosine residues. The activated FGFR2 phosphorylates FRS2 on several sites, allowing the recruitment of the adaptor proteins ‘Son of Sevenless’ (SOS) and growth factor receptor bound 2 (GRb2) to activate RAS and the downstream MAPK pathways (FIG. 2). A separate complex involving GRb2 associated binding protein 1 (GAb1) recruits a complex, which includes PI3K, and this activates a PI3K-AKT signaling pathway. Another FGFR2 binding partner is phospholipase Cγ (PLCγ), which binds at the carboxyl-terminal tail on auto-phosphorylation of FGFR2. After PLCγ is activated, it hydrolyses phosphatidylinositol 4,5 biphosphate (PIP2) to phosphatidylinositol 3,4,5 triphosphate (PIP3) and diacylglycerol (DAG), activating protein kinase C (PKC), which enhances the stimulation of the MAPK pathway by phosphorylating RAF [Chu-Xia Deng 2017].

NOTCH3-HER2 up-regulates the transcription of the Notch pathway components: jagged-1, -2 (Jag-1/2), DLL-1, ADAM17, presenilin-1 (Pres-1), as well as Notch-3 and Notch-1 (Notch-3/1) through the MAPK pathway (FIG. 2). Increased transcription of the Notch pathway components causes Notch-mediated up-regulation of c-Myc, CCND1, phosphorylation of Serine-473 of AKT (+P-Ser 473 AKT), and down-regulation of the tumor suppressor, PTEN [Andrew T. Baker, 2014]. PIK3CA gene is linked to the CCNG1 pathway through PI3k-AKT signaling pathway which increases transcription of CCND1 (FIG. 2).

Brd4—Bromodomain-containing protein 4 (Brd4) is an epigenetic reader and transcriptional regulator recently identified as a cancer therapeutic target for acute myeloid leukemia, multiple myeloma, and Burkitt's lymphoma [Wu et al., 2013]. Via interaction screen and domain mapping, Wu et al. identified p53 as a functional partner of Brd4. Notably, Brd4 association with p53 is modulated by casein kinase II (CK2)-mediated phosphorylation of a conserved acidic region in Brd4 that selectively contacts either a juxtaposed bromodomain or an adjacent basic region to dictate the ability of Brd4 binding to chromatin and also the recruitment of p53 to regulated promoters. In summary, Brd4 interacts directly with multiple DNA-binding proteins and chromatin-modifiers (FIG. 3), Brd4 is a newly identified transcription coactivator for p53 tumor suppressor protein, and CK2-phosphorylated Brd4 is an active chromatin binder and p53 recruiter.

EMSY—Recently, a novel gene, EMSY, has been described providing a new mechanism possibly linking BRCA2 to sporadic breast and ovarian cancer. EMSY is amplified in 13% of sporadic breast cancer and 17% of high-grade ovarian cancer. EMSY (11q13.5) maps to the same amplicon as GARP, glycoprotein A repetitions predominant, a gene not expressed in breast cancer, presenting EMSY as the gene of interest within this amplicon. Amplification of the 11q13 locus is common in several tumor types. Investigation of this gene dense region has led to several candidate targets of amplification. CCND1 and EMS1 (cortactin)—located on separate amplicons at 11q13.3 (CCND1 mapping 0.8 Mb proximal to EMS1)—are considered strong candidate oncogenes within this region and are frequently co-amplified in breast cancer. Other genes in this region include RSF-1, a gene that was recently described as amplified in a subset of ovarian carcinomas. Amplification of EMSY occurs independently of adjacent CCND1, and correlates directly with increased levels of mRNA. The amino terminal of EMSY (ENT-domain) binds to the independent activation domain of BRCA2 encoded by exon 3 of the BRCA2 gene. Deletion of this specific region is known to be the sole mutation in a Swedish breast/ovarian cancer family. Overexpression of EMSY interferes with the activation potential domain of BRCA2 encoded by exon 3 resulting in decreased BRCA2 activity, mimicking the genomic instability phenotype as seen in BRCA2 deficient cells.

In some aspects, the second therapeutic agent may bind HER2, which may, but need not, cause inhibition of Her2 homodimerization, heterodimerization, or phosphorylation. In some aspects, the second therapeutic agent may inhibit the P13K/AKT signaling pathway.

In some aspects, the second therapeutic agent may comprise a monoclonal antibody, that may specifically bind HER2. In some aspects, the second therapeutic agent comprise Trastuzumab or Pertuzumab.

In conjunction with administration of the aforementioned first and second therapeutic agents, additional therapeutic agents that act on other molecular targets, which may but need not be surface expressed biomarkers, may be administered. Thus, in one aspect, the method comprises administering a third therapeutic agent that acts on a molecular target in a biochemical pathway that increases cyclin protein activity, wherein the third therapeutic agent inhibits or reduces one or more activities or products of the biochemical pathway, and wherein the third therapeutic agent acts on a different molecular target than the first and second therapeutic agents. In some aspects, the third therapeutic agent acts on a molecular target selected from the group consisting of ER, PR HER2, AR, FGF13, FGF14, FGF19, PIK3CA, NOTCH3, EMSY, TP52, and BRD4. In some aspects, the third therapeutic agent may bind to a molecular target selected from the group consisting of ER, PR HER2, AR, FGF13, FGF14, FGF19, PIK3CA, NOTCH3, EMSY, TP52, and BRD4.

In some aspects, the third therapeutic agent may inhibit signaling by the ER. In some aspects, the third therapeutic agent may inhibit the production of estrogen. In some aspects, the third therapeutic agent may be an aromatase inhibitor, which may be Letrozole.

In some aspects, the tumor-targeted vector may be DeltaRex-G or DeltaRex-GT. Construction of DeltaRex-GT is disclosed in U.S. Patent Publication US2019/0382459, which is incorporated herein by reference in its entirety (see, in particular, FIG. 3 and corresponding text).

In some aspects, the therapeutic agents may be administered individually at different times or they may be co-administered. In some aspects, the therapeutic agents may be administered as part of a multiple dose regimen, (e.g., sequentially, e.g., on different overlapping schedules with the administration of one or more. In some aspects, the therapeutic agents may be part of a single dosage form, mixed together in a single composition. Moreover, the number of administrations of any particular therapeutic agent may be the same as, or different from, the number of administrations of another therapeutic agent.

As used herein, “treating” means alleviating one or more clinical signs and/or symptoms of cancer. “treating” may comprise, but is not limited to, a decrease in the number of cancer cells, a reduction in tumor size, and/or decrease in the rate of infiltration of cancer cells into peripheral organs (e.g., metastasis). A treatment may achieve a “cure,” that is, a complete and permanent remission of a cancer, but it need not be a cure. Treatment may lead to temporary remission or render the tumor more amenable to other therapeutic options (such as surgery, radiation, or treatment with a different therapeutic agent or combination of agents). It should also be noted that use of these terms is not meant to exclude other steps that may be necessary or desirable for the management and care of a cancer patient but that are not recited in the methods described in this disclosure, e.g., use of IV fluids for the patient's hydration or use of medications to treat pain.

Any type of cancer may be treated using a method of the disclosure. Examples of cancers suitable for treatment using the disclosed methods/include, but are not limited to, breast cancer, colon cancer, liver cancer, lung cancer, bladder cancer, ovarian cancer, testicular cancer, prostate cancer, brain cancer, glioblastoma, esophageal cancer, endometrial cancer, epithelial cancer, stomach cancer, and thyroid cancer. In some aspects, the cancer may comprise a palpable tumor.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLES

### Example 1

This example describes use of a method of the disclosure in the treatment of breast cancer

### Patient and Methods

### Clinical Protocol

Primary objective: To determine the duration of disease-free survival.
Secondary objectives: To determine overall survival at 6 and 12 months and to assess the incidence of treatment-related adverse events.
Exploratory objective: To correlate molecular residual disease (MRD) with duration of disease-free survival.
1. A written informed consent will be obtained prior to treatment.
2. Baseline and follow-up studies. Clinical status, hematologic and organ function, EKG, ECHO, will be assessed within 2 weeks of treatment initiation and prn. CT scan/MRI, PET CT will be obtained prn. Signatera MRD will be obtained every 3 months.
3. Dose, schedule, and route of administration. The vector is stored in a minus 65° C. freezer. Fifteen minutes before infusion, the product is thawed at 37° C. water bath and infused via a peripheral vein or a central intravenous (i.v.) line at 4 ml per minute within one hour upon thawing. DeltaRex-G is given at 1.2-3.6×10¹¹ colony forming units (cfu)/dose three times a week×4 weeks with a 2-week rest period (one treatment cycle), in combination with trastuzumab (trastuzumab pi) at an initial dose of 4 mg/kg as a 90-minute i.v. infusion followed by subsequent once weekly doses of 2 mg/kg as 30-minute i.v. infusions for 18 weeks and every three weeks thereafter up to 52 weeks, and letrozole (letrozole pi) 2.5 mg po daily.
4. If the patient develops a treatment-related >Grade 3 adverse event (CTCAE vs 5.0), the chemotherapy infusions will be held and the patient will be monitored until the toxicity has resolved to <Grade 1, and the patient is stable, after which treatment may be resumed. Since DeltaRex-G is not associated with myelosuppression or organ dysfunction in Phase 1 and 2 clinical trials [Gordon 2004, 2006, 2010, 2011; Chawla 2009, 2010, 2016, 2019, Bruckner 2019]. If the adverse event does not resolve to <Grade 1 within 3 weeks, the combination therapy will be held until the data are discussed with the Food and Drug Administration (FDA) and a decision is made whether to continue or terminate the study.
5. Other Concomitant Medications. Thirty minutes prior to vector infusion: Acute reaction prophylactic therapy consists of diphenhydramine 25 mg p.o. with or without dexamethasone 2 mg i.v. 30 minutes before vector infusion. During or post infusion, dexamethasone 2 mg i.v., diphenhydramine 25-50 mg IV or po may be given for acute hypersensitivity reactions and meperidine 25-50 mg IV may be given for chills. Non-steroidal anti-inflammatory drugs, such as ibuprofen 400 mg q6 hrs. and/or artemisinin 200 mg BID, may be used prn for pain and/or fever.
6. Safety analysis: Baseline (within 2 weeks of treatment) and Follow-up Tests: Medical History, Physical Examination, CBC, CMP to be performed (baseline and q 2 weeks or prn) during the treatment period. EKG, Echo (baseline and as clinically indicated). All serious DeltaRex-G therapy-related or unexpected adverse events will be reported to the FDA and the IRB within 7 days of the incident. All other adverse events will be reported to the FDA and IRB in annual reports and final study report. All Grade III or IV toxicities will be reported in the clinical study summary report.
7. Stopping Rules. The NCI Common Toxicity Criteria (CTCAE version 5.0) will be used to achieve consistency in response to drug/intervention toxicities. Toxicity will be graded on a 1 to 5 grading scale. If the patient develops a DeltaRex-G treatment-related >Grade 3 adverse event (CTCAE Vs 5.0), the DeltaRex-G infusions will be held and the patient will be monitored until the toxicity has resolved to <Grade 1, and the patient is stable, after which treatment may be resumed. If the adverse event does not resolve to <Grade 1 within 3 weeks, the DeltaRex-G treatment will be held until the data are discussed with the Food and Drug Administration (FDA) and a decision is made whether to continue or terminate the study. If the patient develops a trastuzumab-(Trastuzumab pi) or letrozole-(letrozole pi) related >Grade 3 adverse event (CTCAE Vs 5.0), the trastuzumab infusions and/or letrozole will be held and the patient will be monitored until the toxicity has resolved to ≤Grade 1, and the patient is stable, after which treatment may be resumed. If the adverse event does not resolve to <Grade 1 within 3 weeks, the chemotherapy will be held until the data are discussed with the Food and Drug Administration (FDA) and a decision is made whether to continue or terminate the study.
8. Efficacy analysis: Treatment outcome parameters include duration of disease-free survival and overall survival. Mammogram/Ultrasound of breast at 6 and 12 months, MRI, PET CT scan, when clinically indicated.
9. Exploratory analysis: Molecular residual disease (MRD) using Signatera ctDNA and tumor markers will be obtained every 3 months for detection of early recurrence and correlation with overt recurrence/metastasis [Coombes 2019].

### Results

### Case Study

A 75-year-old female, who was in her usual state of health until Nov. 20, 2020 incidentally noticed a mass over upper quadrant of left breast with no nipple discharge or retraction, overlying skin changes nor lymphadenopathy. Patient has been on hormonal replacement since going through menopause 20+ years ago. She underwent a screening mammogram followed by ultrasound confirming a left upper quadrant breast mass @ 2o'clock position suspicious for malignancy. Histopathological examination performed on Nov. 24, 2020 of five core biopsies showed ER+PR+, HER 2 amplified (3+) poorly differentiated invasive ductal carcinoma with Ki-67 of 15% and up to 30% in some spots. Patient underwent MRI of the breasts on Nov. 30, 2020 which showed a solitary 1.6 cm spiculated left breast mass corresponding to biopsy-proven malignancy, without suspicious adenopathy. On Dec. 4, 2020 patient underwent left breast partial mastectomy with sentinel lymph node resection Surgical pathology report confirmed the diagnosis of invasive ductal carcinoma, poorly differentiated, tumor size of 1.7×1.6×1.5 cm, ER+PR+, AR+, HER2 amplified with sentinel lymph node positive for isolated tumor cells (0.06 mm tumor present) (T1c, N1).

On further evaluation, molecular profiling revealed amplification of the following genes-CCND1, ERBB2, FGF4, FGF19, BRD4, FGF3, EMSY, NOTCH3 with TP53 and PIK3CA mutations, indicating chemotherapy resistance and poor prognosis [Baker 2014; Baker 2019; Buendia 2020; Cheek 2004; Deng 2017; Emi 2008; Federova 2020; Lee 2013; Li 2008; Taneja 2010; Mackay 2011; Mohammadizadeh 2013; Montaudon 2020; Pradeep 2011]. The oncogenic drivers found in this patient's molecular profile represented proximal oncogenic mechanisms that activate the CCNG1 pathway (see FIGS. 2 and 3), indicating that inhibition of the CCNG1 pathway would be a viable adjuvant/first line treatment option for this patient. Based on her molecular profile, patient opted to receive DeltaRex-G.

On Dec. 21, 2020, patient started on trastuzumab. On Dec. 28, 2020, the patient started Letrozole and on Jan. 4, 2021, the patient started Delta Rex-G regimen. The patient received DeltaRex-G i.v. (1.2-3-6×10e11 cfu/dose) three times a week in combination with trastuzumab weekly and letrozole po daily.

To date, the patient has received 48 dose infusions of DeltaRex-G, 30 doses of trastuzumab and 401 doses of letrozole with no treatment related adverse reactions and no evidence of recurrence since diagnosis (12 months), with a persistently negative Signatera MRD test, an assay for the detection of molecular residual disease (MRD) or circulating tumor DNA (ctDNA) in patient plasma, recurrence monitoring and treatment response evaluation. In previous breast cancer studies [Coombes 2019], a negative result indicated that there were no ctDNAs detected, predicting less chance of recurrence or a favorable response to an ongoing treatment. The combination treatment with DeltaRex-G, trastuzumab and letrozole is on-going.

### Discussion

The co-expression of hormone receptors and HER2 IBC results in a higher proliferation index as compared to ER positive, HER2 negative IBC. There are distinct biologic differences among HER2+ tumors according to the expression of hormone receptors. In HER2+/HR+ breast cancer, about two-thirds of triple receptor positive cases are Luminal A or B indicating estrogen receptor-dependency, whereas about 75% of HER2+/HR− cases are HER2-amplified and only 10% Luminal A or B. Immune microenvironment may also be different in HER2+/HR+ vs HER2+/HR-tumors. The level of stromal infiltrating lymphocytes is lower in HER2+ tumors co-expressing hormone receptors as compared to HER2+/HR− tumors. These biological differences account for a different prognostic pattern and treatment sensitivity. In 2020, Dieci et al reported that HER2+/HR+ breast cancer patients have a reduced sensitivity to neoadjuvant chemotherapy+anti-HER2 agents [Dieci, 2020] and that HER2+/HR+ patients deserve more personalized treatment options. And given the reduced chemosensitivity of HER2+/HR+ breast cancer to chemotherapy, there is increasing interest in the development of effective chemotherapy-free therapeutic strategies, by taking advantage of targeting the estrogen receptor, the HER2 receptor pathways and the patient's unique molecular profile involving oncogenic drivers of signaling pathways of cellular proliferation in IBC [Dieci, 2020].

The patient's molecular profile reports showed that in addition of the tumor expressing ER+, PR+, AR+and HER2 (ERB2) amplification, the following oncogenes were also amplified: CCND1, FGF4, FGF19, BRD4, FGF3, EMSY, NOTCH3. Mutations in DNA and protein of PIK3CA (c.3140A; p.H1047R) and TP53 (c.995T>C; p.1332T) respectively were also identified further indicating chemotherapy resistance/poor outcome [Baker 2014; Baker 2019; Buendia 2020;Cheek 2004; Deng 2017; Emi 2008; Federova 2020; Lee 2013; Li 2008; Taneja 2010; Mackay 2011; Mohammadizadeh 2013; Montaudon 2020; Pradeep 2011]. Of interest, these the oncogenes amplified in the patient's tumor represent proximal oncogenic mechanisms that activate the CCNG1 pathway, suggesting that inhibition of the CCNG1 pathway would be a viable adjuvant/first line treatment option for this patient.

Cyclin G1 (CCNG1) is a “non-canonical” plausibly oncogenic “cyclin-like” protein whose action and expression is among the earliest cell cycle events that drive the normally quiescent stem cell from quiescence (i.e., G0); biochemically gaining the cellular “Competence to Proliferate” by assembling an executive set of oncogenes needed to enter the G1 phase of the cell division cycle. Moreover, the Axis of Cyclin G1-associated enzymes and oncogenes (Cdk2, Myc, Mdm2, P53, p18 Hamlet) operate throughout the cell cycle to ensure cell survival with DNA Fidelity in the balance. [Hall 2021; Gordon 2021]. Cyclin G1 physically binds to a major cellular ser/thr protein phosphatase subunit designated 2A (PP2A), thereby “targeting” the otherwise undiscerning phosphatase activity to a cyclin G1-targeted protein, Mdm2 (oncogene product). The Mdm2 protein, in turn, targets, inhibits, and degrades the p53 tumor suppressor (guardian of DNA fidelity with executioner functions) in the normal regulation of the cell division cycle. Also, Cyclin G1 partners with CDK2/CDK5 to phosphorylate (activate/stabilize) the c-Myc oncoprotein, which in turn provides the transcriptional drive for selective protein synthesis at the very threshold of the G0 to G1 transition and beyond [Ahmad Al-Shihabi, 2018; Hall 2021, Gordon 2021] . The oncogenic pathway (i.e., the Cyclin G1/Cdk2/Myc/Mdm2/p53 Axis) is distinguishable from the set of so-called canonical G1 cyclins (D1, D2, D3, cyclin E, cyclin A) that target CDK complexes cyclically and precisely to pRB (and Rb-related) tumor suppressor proteins, whose inhibition releases suppression of E2F-family of transcription factors and regulatory events which drive G1-phase cells to irreversibly enter the S-phase of the division cycle (G1 to S) (Cyclin/CDK/Rb/E2F axis) [Ahmad Al-Shihabi, 2018; Hall 2021; Gordon 2021].

### Conclusion

In summary, this is the first FDA authorized treatment protocol for triple receptor positive IBC using DeltaRex-G, a tumor-targeted retrovector encoding a CCNG1 inhibitor gene, in combination with trastuzumab and letrozole. The first patient to receive this combinatorial therapy is a 75 year-old female with early-stage IBC and multiple oncogene amplifications and mutations relevant to CCNG1 and the emerging Cyclin G1/Cdk2/Myc/Mdm2/p53 Axis of cell competence/survival. The patient has received 48 infusions of DeltaRex-G, 20 infusions of trastuzumab and 154 doses of letrozole with no adverse reactions, is in remission with negative Signatera MRD 6 months from diagnosis.

### Based on These Results:

DeltaRex-G may be a viable adjuvant/first line therapy for post-menopausal women with early-stage triple receptor positive invasive breast cancer.
Combinatorial therapy with DeltaRex-G, trastuzumab and letrozole has no additive toxicity.
A Phase 2 clinical trial is planned to evaluate if DeltaRex-G is equally effective (not-inferior-to) than standard chemotherapy/radiation therapy as adjuvant/first line therapy for early-stage hormone receptor positive and HER2 amplified breast cancer.

The contents of US20210299276, Methods of Exploiting Oncogenic Drivers Along the Human Cyclin G1 Pathway for Cancer Gene Therapy, are incorporated herein by reference in their entirety.

What is claimed is:

1. A method of treating a cancer in an individual, wherein the cancer cells express at least one biomarker that is part of a biochemical pathway that promotes cyclin protein activity, the method comprising administering to the individual:
a. a first therapeutic agent comprising a tumor-targeted vector encoding a cytocidal inhibitor of cyclin 1; and,
b. a second therapeutic agent that acts on the biochemical pathway that increase cyclin protein activity;
wherein the second therapeutic agent inhibits the biochemical pathway.

2. The method of claim 1, wherein the second therapeutic agent binds the at least one biomarker.

3. The method of claim 1or 2, wherein the biochemical pathway comprises an oncogenic receptor-mediated signaling pathway.

4. The method of claim 1 or 2, wherein the biochemical pathway comprises a tumor suppressor pathway.

5. The method of claim 1 or 2, wherein the biochemical pathway comprises a stem cell renewal or differentiation pathway.

6. The method of any one of claims 1-5, wherein the at least one biomarker is selected from the group consisting of estrogen receptor (ER), progesterone receptor (PR) human epidermal growth factor 2 (HER2), androgen receptor (AR), fibroblast growth factor 13 (FGF13), FGF14, FGF19, phosphatidylinositol 3-kinase catalytic subunit alpha (PIK3CA), NOTCH3, EMSY, tumor protein 53 (TP52), and bromodomain-containing protein 4 (BRD4).

7. The method of any one of claims 1-6, wherein the second therapeutic agent binds HER2.

8. The method of claim 7, wherein the second therapeutic agent inhibits HER2 heterodimerization.

9. The method of claim 7 or 8, wherein the second therapeutic agent inhibits the P13K/AKT signaling pathway.

10. The method of any one of claims 1-9, wherein the second therapeutic agent comprises a monoclonal antibody that specifically binds HER2.

11. The method of any one of claims 1-10, wherein the second therapeutic agent comprises Trastuzumab or Pertuzumab.

12. The method of any one of claims 1-11, wherein the cancer cells express at least two or more biomarkers, each of which is part of a unique biochemical pathway that promotes cyclin protein activity, wherein the method comprises administering to the individual a third therapeutic agent that inhibits a biochemical pathway, and wherein the second and third therapeutic agents inhibit different biochemical pathways.

13. The method of claim 12, wherein the third therapeutic agent inhibits signaling by the ER.

14. The method of claim 13, wherein the third therapeutic agent inhibits the production of estrogen.

15. The method of claim 13 or 14, wherein the third therapeutic agent is an aromatase inhibitor.

16. The method of any one of claims 12-15, wherein the third therapeutic agent is Letrozole.

17. The method of any one of claim 1-16, wherein the tumor-targeted vector is DeltaRex-G or DeltaRex-GT.

18. The method of any one of claim 1-17, wherein the cancer in the individual includes a breast cancer.

19. The method of claim 18, wherein the individual is a post-menopausal woman.

20. The method of claim 18 or 19, wherein the breast cancer is an early-stage triple receptor positive invasive breast cancer.
